(19) United States
(12) Patent Application Publication
(10) Pub. No.: US 2026/0137582 A1
(43) Pub. Date: May 21, 2026

(54) DEVICE AND METHOD FOR OPENING AN AIRWAY

(71) Applicant: SOMMETRICS, INC., San Diego, CA (US)

(72) Inventor: Kent GANDOLA, San Diego, CA (US)

(73) Assignee: SOMMETRICS, INC., San Diego, CA (US)

(21) Appl. No.: 18/705,942

(22) PCT Filed: Oct. 26, 2022
(86) PCT No.: PCT/US2022/047857

§ 371 Date: Apr. 29, 2024

Related U.S. Application Data
(60) Provisional application No. 63/263,328, filed on Oct. 29, 2021.

(51) Int. Cl. A61H 9/00; A61F 5/56; A61M 1/00

(52) U.S. Cl. CPC A61H 9/0057; A61F 5/56; A61M 1/985; A61H 2201/1207; A61H 2201/1238; A61H 2201/1604; A61H 2201/1609; A61H 2201/165; A61H 2205/04

(57) ABSTRACT

The present invention provides devices and methods for creating and/or maintaining a seal between a pump element and a vacuum chamber. The vacuum chamber device is configured to fit on the skin of a subject, for example at an external location corresponding approximately with the subject's internal soft tissue associated with the neck's anterior triangle. The sealing device includes structural elements designed to optimize ease of use for the user while providing optimal sealing of device components, achieved through features that avoid excessive forces during assembly and disassembly while maintaining the integrity of the chamber device shape and air-tight seals during operation of the device.

### CROSS-REFERENCE TO RELATED APPLICATIONS

The present application claims the benefit of priority to U.S. Provisional Application No. 63/263,328, filed on Oct. 29, 2021, the contents of which are incorporated herein by reference.

### BACKGROUND OF THE INVENTION

The following discussion of the background of the invention is merely provided to aid the reader in understanding the invention and is not admitted to describe or constitute prior art to the present invention.

U.S. Pat. Nos. 5,343,878, 7,182,082, 7,762,263, 9,655,766, 10,780,017, and 10,874,577 describe various devices which purport to utilize external application of negative pressure upon the external neck surface of patients. A therapeutic appliance is typically provided that has a surface which is configured to enclose an external area of the neck overlying a portion of the upper respiratory passage. In certain embodiments, these appliances can provide a chamber body (e.g., a hollow space filled with air molecules) lying between the interior surface of the chamber body and the throat. Therapy appliance is operably connected to an air pump which is configured to produce a partial vacuum in this chamber body. Application of a therapeutic level of negative pressure in the chamber body elicits movement of the upper airway and may alleviate conditions such as snoring, sleep apnea, and airway obstruction such as a full or partial airway collapse for example.

In these “negative pressure” therapeutic apparatuses and methods it can be difficult to obtain a proper fit between elements of the apparatus for an air-tight seal that also accommodate ease of assembly and disassembly. For example, the fit between the chamber body of a therapy appliance and an air pump (that is designed to create and maintain the differential negative pressure, relative to atmospheric pressure for example, at the desired location on the patient). This is particularly true for the devices that are intended for daily wear for many hours and subject to frequent disassembly and assembly of components for cleaning, for example. Therefore, the success of these negative pressure therapies is optimized by a device's ability to accommodate (flex, bend, flow, etc.) varying anatomical features (i.e. device compliance) through a range of motion, while also having structural elements to support elements of the device and structural elements that allow for ease of assembly and disassembly by the user without compromising a leak-free seal of device and device components during use. User compliance with therapy thereby being maximized by ease of assembly and disassembly of elements of the device, a good comfortable interface between the device and the user and the device and the leak-free seal of components of the device should accommodate movement to different sleeping positions without loss of seal between the elements of the device as well of loss of seal between the device and the user.

### BRIEF DESCRIPTION OF THE INVENTION

It is an object of the invention to provide a sealing system for attaching an air pump element to a therapy device which has a sealed chamber body that is intended to attach and seal to a patient's external tissue, such as a face, a neck, an area surrounding a wound, etc. wherein the sealed chamber body has a sealable aperture through which the air pump element is affixed. This therapy device is particularly suited for forming a sealed chamber body that is configured for the administration of negative pressure to a targeted therapy on the external tissue of an individual. By so doing, the external negative pressure on the skin impacts the underlying tissues such as the tongue and other tissues surrounding the upper airway in the neck. Therapy device of the invention is preferably configured to be a completely body-worn system.

In a first aspect, the invention provides therapy devices configured for the administration of negative pressure to skin tissue of an individual. These therapy devices comprise:
a chamber body comprising
   (i) a skin interface at the periphery of the chamber body configured to provide a skin contact surface with the individual, wherein when the skin interface is in contact with the individual, the chamber body defines an external aspect facing the ambient environment, an internal aspect facing the skin tissue, and an enclosed interior volume overlying the skin tissue that is enclosed by the chamber body,
   (ii) an aperture through the chamber body,
   (iii) a sealing interface at the periphery of the aperture, the sealing interface comprising
      a lip seal comprising a resilient sealing lip projecting toward the interior of the chamber body,
      a first recess posterior to the resilient sealing lip relative to the aperture,
      a first backing support posterior to the first recess relative to the aperture,
      a second recess posterior to the backing support relative to the aperture, and
      a second backing support posterior to the second recess relative to the aperture,
      wherein the lip seal and the first and second backing supports are configured as unitary components of the chamber body;
an air pump configured to insert into the aperture from the external aspect of the chamber body and comprising a first collar interface surface, wherein upon insertion into the aperture the air pump is operably connected to the chamber body to remove air from the enclosed volume when energized, the air pump deflects the resilient sealing lip into the first recess, thereby forming an airtight seal with the chamber body, and the first collar interface surface is accessible from the interior of the chamber body;
a rigid or substantially rigid retainer collar comprising a continuous peripheral edge region configured to insert into the second recess and a second collar interface surface configured to cooperatively engage with the first collar interface surface from within the interior aspect of the chamber body, wherein insertion of the peripheral edge region into the second recess and engagement of the first and second collar interface surfaces reversibly retains the air pump and retainer collar to the chamber body.

In certain embodiments, therapy devices of the present invention are configured and arranged for producing a region of negative pressure over tissues of the anterior triangle of the neck, e.g., for purposes of treating conditions such as sleep apnea, snoring, or airway obstruction. Such devices may be referred to as negative external pressure (NEP) therapy devices to distinguish from positive airway pressure (PAP) therapy devices. In these embodiments, a therapy device can be configured such that the skin interface approximately conforms to a continuous contact area on the individual defined by a first location approximately corresponding to a first gonion on one side of the individual's mandibular body, a second location approximately corresponding to the individual's mental protuberance, a third location approximately corresponding to a second gonion on the opposite side of the individual's mandibular body, and a fourth location approximately corresponding to the individual's thyroid cartilage. Alternatively, such a therapy device can be in the form of a continuous collar such as disclosed in U.S. Pat. No. 10,780,017B2, which is hereby incorporated by reference.

In other embodiments, therapy devices of the present invention are configured and arranged for producing a region of negative pressure over a wound. Such devices may be referred to as negative pressure wound therapy (NPWT) devices. See, e.g., WO2013175306, WO2016174048, WO2017153357, WO2018060417, WO2019129581, and WO2020187971, each of which are hereby incorporated by reference.

In certain embodiments, the lip seal is positioned at or very near to the external aspect of the chamber body. In most preferred embodiments, the resilient sealing lip contacts the air pump when the air pump is seated into the aperture, but the resilient sealing lip does not contact the retainer collar when the peripheral edge region is seated into the second recess. In certain embodiments, the terminal portion of the resilient sealing lip comprises a radiused edge at the point of contact with the air pump.

In certain embodiments the first and second collar interface surfaces cooperate to provide a reversible locking mechanism that orients the air pump relative to the to the retainer collar. By way of an example, the first and second collar interface surfaces can be in the form a cooperative twist coupling such as by providing a first bayonet connector on the air pump and a second, corresponding second bayonet connector on the retainer collar. Rotation of one of the bayonet connectors relative to the other causes a pin on one bayonet connector to engage with a corresponding slot on the other bayonet connector which acts as a stop and a lock until the pin is pushed out of the slot.

In preferred embodiments, the second recess will extend between the first backing support and the second backing support to at least the bending moment of the resilient sealing lip and more preferably beyond the bending moment of the resilient sealing lip. In these embodiments, seating of the retainer collar preferably causes the continuous peripheral edge region to insert into the second recess until the second recess is fully occupied by the continuous peripheral edge region.

In certain embodiments, the air pump and/or the retainer collar comprise mount points that engage with receiving points on the chamber body to both secure the pump module to the chamber body, and do so in a specific orientation. By way of example only, posts, tabs, or other similar structural features can be provided on the retainer collar that are configured to insert into corresponding slots on the internal aspect of the chamber body. The retainer collar may be held to the collar by the frictional component of the interaction between the structural features and the corresponding slots. The air pump may then be inserted into the aperture and, through rotation of the air pump, be affixed to the retainer collar using a mechanism (e.g., a bayonet mount) which limits the air pump to a specific reversibly locked orientation on the retainer collar. The skilled artisan will understand that this arrangement may be reversed, and the structural features provided on the chamber body that engage corresponding slots on the retainer collar. Alternatively, the structural features may be provided on the air pump and the corresponding slots on the external aspect of the chamber body. This discussion is exemplary only, and one of skill in the art can readily identify similar means for securing the pump module to the chamber body in a specific orientation.

In one embodiment, the retainer collar comprises mount points that engage with receiving points on the chamber body to secure the air pump to the chamber body, while the air pump and retainer collar comprise thread and groove elements to enable a twist coupling between the air pump to the retainer collar until reversibly arresting and locking against the chamber body and to do so with no specific end-orientation of the air pump.

In another embodiment, the retainer collar comprises mount points that engage with receiving points on the chamber body to secure the air pump to the chamber body, while the air pump and retainer collar comprise mechanical (e.g. detent or cam) or magnetic means to resist or arrest insertion and/or rotation of the air pump and to do so to a reversibly locked orientation relative to the retainer collar.

As used herein, the term “rigid” means: for rubbers, plastics, and other non-metallic materials that are subjected to hardness testing according to ASTM D-2240, a Shore D hardness of at least 75 according to ASTM D-2240, and for other materials, a hardness value according to a standardized method providing protection for the user equivalent to the Shore D hardness discussed in this sentence. As used herein, the term “substantially rigid” means: for rubbers, plastics, and other non-metallic materials that are subjected to hardness testing according to ASTM D-2240, a Shore D hardness of at least 60 according to ASTM D-2240, and for other materials, a hardness value according to a standardized method providing protection for the user equivalent to the Shore D hardness discussed in this sentence. Thus, the term “substantially rigid” would include, for example, rubbers, plastics, and other non-metallic materials that are subjected to hardness testing according to ASTM D-2240, a Shore D hardness of at least 65 according to ASTM D-2240.

Such rigid or substantially rigid materials may comprise, for example, polymer materials such as polypropylene, polyethylene, polybutylene terephthalate, acrylonitrile butadiene styrene (“ABS”), polycarbonate (“PC”), and ABS/PC alloys. Desired properties of the rigid or substantially rigid materials include, for example, one or more of hardness, toughness, low density (i.e., light weight), and inertness (to minimize skin irritations, rashes, etc.). As a result, the one or more rigid or substantially rigid materials could comprise, for example, at least one of: metals; natural or synthetic rubber compounds with reinforcing fibers (such as Kevlar® pulp); or Kevlar®. The one or more rigid or substantially rigid materials may incorporate advances from the nanotechnology field. In one embodiment, cage member 102 comprises one substantially rigid material, Thermoplastic Polyurethane (“TPU”).

The terms “external area” and “external surface” of an individual as used herein refers to a portion of the external skin surface. Target tissues for negative pressure therapy may be the tissues underlying this external surface, and can include upper respiratory passages and associated tissues, a wound in the external surface, etc. In various embodiments, therapy device is configured to provide optimized fitting parameters, for example, seal, comfort and local device compliance throughout all anatomical points of contact selected to accommodate the targeted body location.

Any and all air pump types find use in the present invention, provided that a therapeutic level of vacuum can be achieved by the pump. Preferred therapeutic vacuum levels are in a range of between about 7.6 cm H₂O to about 61 cm H₂O, and preferably between about 20 and about 45 cm H₂O. In certain embodiments, the air pump comprises a piezoelectric material configured to provide an oscillatory pumping motion. It is preferred that the oscillatory pumping motion operates at a frequency greater than 500 Hz and most preferably at a frequency greater than 20,000 Hz, i.e. beyond the frequency range of most human hearing. In certain embodiments the vacuum source may be used in a variable manner to maintain therapeutic level of vacuum within a specified range rather than a single value.

In certain embodiments, therapy device comprises one or more vent elements configured to provide a controlled airflow into the chamber body when the chamber body is mated to the individual and therapeutic level of negative pressure is applied within the chamber body. This airflow is preferably between about 10 mL/min and about 200 mL/min, and most preferably between about 20 mL/min and about 100 mL/min, and still more preferably between about 40 mL/min and about 80 mL/min. In one embodiment, the vent element can comprise an aperture and optionally a filter element within the aperture, wherein the filter element comprises a pore size of about 0.25 μm or less, such as a pore size of about 0.1 μm. The aperture may be provided as a feature of the chamber body itself, or may be a feature of the air pump. The filter element can be configured as a replaceable element. The level of airflow can be maintained as a constant value. Alternatively, the level of airflow can vary.

In various embodiments, the vent element can comprise a critical orifice, either within the chamber, or the air pump, or both, wherein the critical orifice enables a flow rate of about 10 to 60 mL/min, and more preferably 30 to 50 mL/min at a target negative pressure value of 30 cm H₂O. In certain embodiments, the level of airflow tied to therapeutic level of vacuum; that is, a higher level of vacuum can be accompanied by a higher level of airflow due to the differential in pressure between the atmospheric side of the vent elements and the interior of the chamber body.

For purposes of the present application, the term “about” and or “approximately” refers to +/−10% of any given value, more preferably +/−5%, and most preferably +/−1%.

### BRIEF DESCRIPTION OF THE DRAWINGS

FIG. 1 is an exploded view of an illustrative embodiment of the device including chamber body 100, the interior aspect of the chamber body 103, the external aspect of chamber body facing the ambient air 105, the skin interface surface designed to make contact with the perimeter of therapy site of the user 107, the lip seal element 109, sealable aperture 110, the retaining element securing recesses 111, the air pump element 120, the sealing surface of the pump element 123, the pump element bayonet feature 125, a rigid or substantially rigid retainer collar 130, comprising, chamber body support feature(s) 133, retaining element mount point features in the form of posts 135 and continuous peripheral edge region 137 providing a lip seal reinforcing element.

FIG. 2 is a rear view of an illustrative embodiment of the chamber body 100 showing the interior aspect 103 of the chamber body, the external aspect 105 of chamber body facing the ambient air, the skin interface designed to make contact with the perimeter of therapy site of the user 107, the lip seal element 109, recesses 111 configured to receive the retaining element mount point posts 135, channel 113 configured to receive and engage the retainer collar, and the sealable aperture element 110.

FIG. 3. Is a front view of and illustrative embodiment of the device including the chamber body 100, the external aspect 105 of the chamber body facing the ambient air, lip seal element 109, sealable aperture 110 and the recesses 111 configured to receive the retaining element mount point posts 135.

FIG. 4. Is a rear view of an illustrative embodiment of a retainer collar 130, comprising chamber body support feature(s) 133, mount points 135, and bayonet feature 139 comprising bayonet stop 140. The rear aspect of the retainer collar is that aspect of the retainer collar that is distal to the aperture and the air pump.

FIG. 5. Is a front view of an illustrative embodiment of a retainer collar 130, comprising chamber body support feature(s) 133, mount points 135, cylindrical peripheral edge region 137, and bayonet feature 139 comprising bayonet stop 140. The front aspect of the retainer collar is that aspect of the retainer collar that is proximal to the aperture and the air pump.

FIG. 6. Is a rear view of an illustrative embodiment of an air pump 120 comprising a pump housing, a sealing surface of the housing 123, and bayonet feature 125 comprising bayonet detent 127.

FIG. 7. Is a sectional view of an illustrative embodiment showing the chamber body 100, the relative location of sealable aperture 110, the interior aspect of the chamber body 103, the exterior aspect of chamber body facing the ambient air 105, a lip seal element comprising resilient sealing lip of lip seal 109, the first recess 117 posterior to the resilient sealing lip, the first backing support 157, the second recess 113 (shown filled by the continuous peripheral edge region of the retainer collar), the second backing support 159, lip seal contact surface 115, apex of first recess 119, and retainer collar 130 comprising continuous peripheral edge region 137 inserted into the second recess.

FIG. 8. Is a sectional view of an illustrative embodiment as in FIG. 7, in which the air pump 120 installed in the aperture 110 of the chamber body 100.

FIG. 9. Is an illustrative embodiment of the invention showing a horizontal section of the chamber body 100 the interior aspect of the chamber body 103, the exterior aspect of chamber body facing the ambient air 105, the surface designed to make contact with the perimeter of therapy site of the user 107, the lip seal element 109, sealable aperture 110, recesses 111 configured to receive the retaining element mount point posts 135, and the second recess 113.

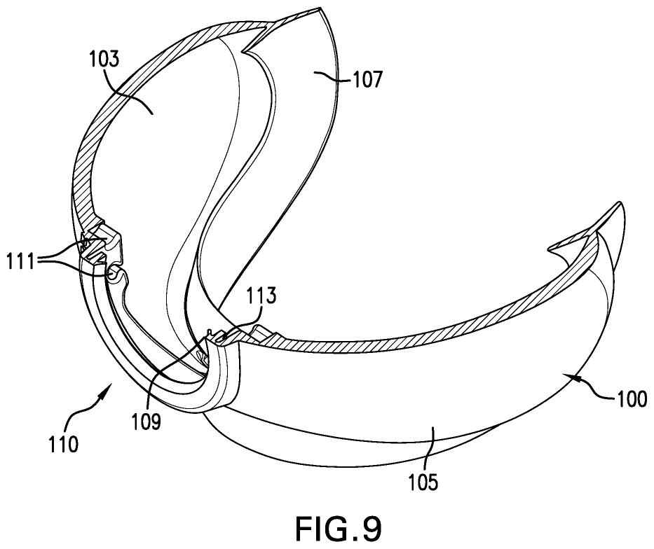

FIG. 10. Is an illustrative embodiment of the invention showing a horizontal section of the chamber body 100 the interior aspect of the chamber body 103, the exterior aspect of chamber body facing the ambient air 105, the lip seal element 109, the sealable aperture 110, recesses 111 configured to receive the retaining element mount point posts 135, the first recess 117, the first backing support 157 and the second backing support 159 defining the second recess 113

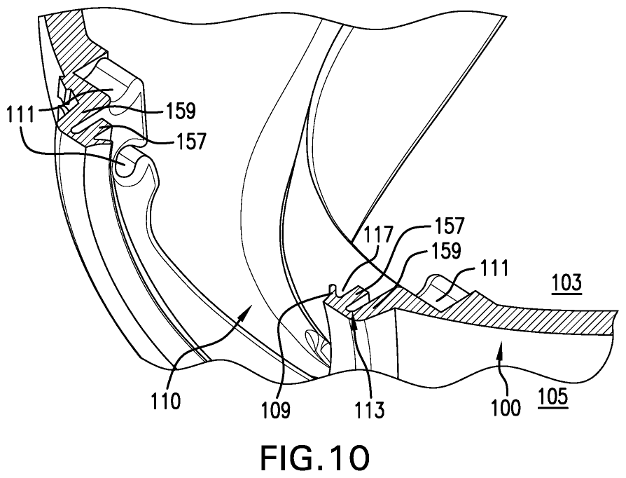

FIG. 11a. is an illustrative embodiment of the invention showing a horizontal section of the chamber body 100 (without the air pump 120) including the interior aspect of the chamber body 103, the exterior aspect of chamber body facing the ambient air 105, the sealable aperture 110. Also shown is a detail 215 further showing the resilient sealing lip of lip seal 109, the retaining element 137, lip seal contact surface 115, first recess 117, apex of the first recess 119, retainer collar 130 the first backing support 157 and the second backing support 159 defining the second recess 113.

FIG. 11b. is an illustrative embodiment of the invention of a closer view of the detail 215 showing a horizontal section of the chamber body 100 (without the air pump 120 installed) including the retainer collar 130. Also shown are the width dimension 140 of the second recess (described hereinafter), radiused point of the lip seal contact surface 141, dimension 143 the distance by which the second recess 113 which ends in a radius 150 (and so the continuous peripheral edge region 137 of the retainer collar 130) extends beyond the apex 119 of lip seal 109 which has an apex radius 153, width dimension 145 of the first recess measured from the apex of the lip seal radius to the pump contact point on lip seal 109, the width dimension 149 of the of the resilient sealing lip of lip seal 109, the angle of lip seal 109 in the uncompressed state (i.e., prior to insertion of air pump 120) 151 the first backing support 157 and the second backing support 159 defining the second recess 113.

FIG. 12. is an illustrative embodiment of the invention as shown in FIG. 11b showing a horizontal section of the chamber body 100, with the air pump 120 installed through the sealable aperture, depicting the compressed dimension 155 of the lip seal when the pump housing is installed.

### DETAILED DESCRIPTION OF THE INVENTION

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

In the present invention, an air pump connection sealing system is designed for a negative pressure therapy device that maximizes ease of use, (i.e. assembly and disassembly) and seal efficiency, ultimately optimizing device efficacy and user compliance. While described below for use in the opening of the upper airway when placed upon the neck of a subject over a surface corresponding to approximately the upper airway of the subject, this exemplary application of the technology is not meant to be limiting.

A typical therapy device of the present invention comprises a chamber body and a skin interface that is preferably unitary to the edge of the chamber body along the circumferential dimension of the skin interface to form an airtight junction between the skin interface and the chamber body and an air pump element attached to the chamber body through a sealable aperture in the chamber body. Thus, the chamber body forms the air-filled dome/chamber of therapy device. The chamber body is preferably flexible in order to adapt to the contours of a variety of anatomical locations. Non-limiting examples of materials suitable for constructing the chamber body include plastics, metals, and resilient materials such as silicone, rubber, or urethane.

An adhesive, a foam, or some other soft material may be located on the contact surface of the skin interface. These elements are configured to maintain an approximate uniform contact pressure with minimized pressure variations along the skin of an individual through all points of contact of therapy device on a patient. By “minimized pressure variation” means a pressure at any point between the contact surface of the skin interface and the patient's tissue varies by no more than about 20%, and preferably no more than about 10% or about 5%, from the average pressure across the entire contact surface. The outer contact surface, as used herein, is the surface of the skin interface of therapy device that makes contact with the skin of the individual forming the contact and sealing surface of therapy device.

In the exemplary embodiment described herein, the skin interface is configured to follow the contour of therapy device, which itself is configured to approximately conform to an individual from approximately a first location corresponding to a first gonion on one side of the individuals mandibular body to a second location corresponding to the individuals mental protuberance to a third location corresponding to the second gonion on the opposite side of the individual's mandibular body and a fourth location corresponding to the individuals thyroid cartilage further configured return to approximately the first location corresponding to the first gonion. “Gonion,” as used herein, describes the approximate anatomical location on each side of the lower jaw on an individual at the mandibular angle. The “mental protuberance,” as used herein, describes the approximate anatomical location of the eminence of the chin, the center of which may be depressed but raised on either side forming the mental tubercles. The “thyroid cartilage,” as used herein, describes the approximate anatomical location of the large cartilage of the larynx in humans.

The term “seal” as used in this context is not to necessarily imply that a perfect seal is formed between therapy device and the contact surface of the individual. Rather, a “seal” is a portion of the device which mates to the wearer and maintains a therapeutic level of vacuum. A certain amount of leakage at the seal may be tolerated so long as the desired negative pressure can be achieved and maintained. Preferred operational vacuum levels are in a range of between 7.6 cm to about 61 cm of water. Preferred forces applied to the user's neck tissues in order to assist in opening the upper airway passages are in a range of about 0.5 kilogram to about 6.68 kilograms. The term “about” and “approximately” as used herein with regard to any value refers to +/−10% of that value.

The dome/chamber enclosed by the chamber body provides a finite volume which is evacuated by the air pump to deliver the desired therapeutic level of partial vacuum. Once generated, the partial vacuum will decay at a rate which results from leakage of air into the chamber body past the seal, and/or through features integrated into the dome or into the air pump to provide a controlled level of air leakage. In certain embodiments, the chamber body encloses a volume of between 0.5 and 12 in³. Preferably, the leakage is no more than between 0.005 and 0.5 in³/min (0.082 and 8.194 mL/min), and most preferably between about 0.01 and 0.1 in³/min (0.164 and 1.639 mL/min).

Therapy device may comprise one or more vent elements. As used herein a vent element is an aperture through therapy device that provides airflow in to the chamber body when the chamber body is mated to the individual and a therapeutic level of negative pressure is applied within the chamber body. The aperture(s) can be in any suitable location on the device. In some embodiments the aperture(s) may be located at the top of the chamber body closer to locations one and three on the individual. In another embodiment, the aperture may be located within the air pump. The vent element(s) may simply be a fixed critical orifice such that when the chamber body is mated to the individual and a therapeutic level of negative pressure is applied an airflow between about 10 mL/min and about 60 mL/min is achieved or an aperture through which a filter element can be inserted to create filtered airflow such that when the chamber body is mated to the individual and a therapeutic level of negative pressure is applied an airflow between about 10 mL/min and about 60 mL/min is achieved. The filter element can be a replaceable element and comprise a pore size of between about 0.25 μm and 0.1 μm or less such that when the chamber body is mated to the individual and a therapeutic level of negative pressure is applied an airflow between about 10 mL/min and about 60 mL/min is achieved. In certain preferred embodiments the airflow is between about 30 mL/min and about 50 mL/min.

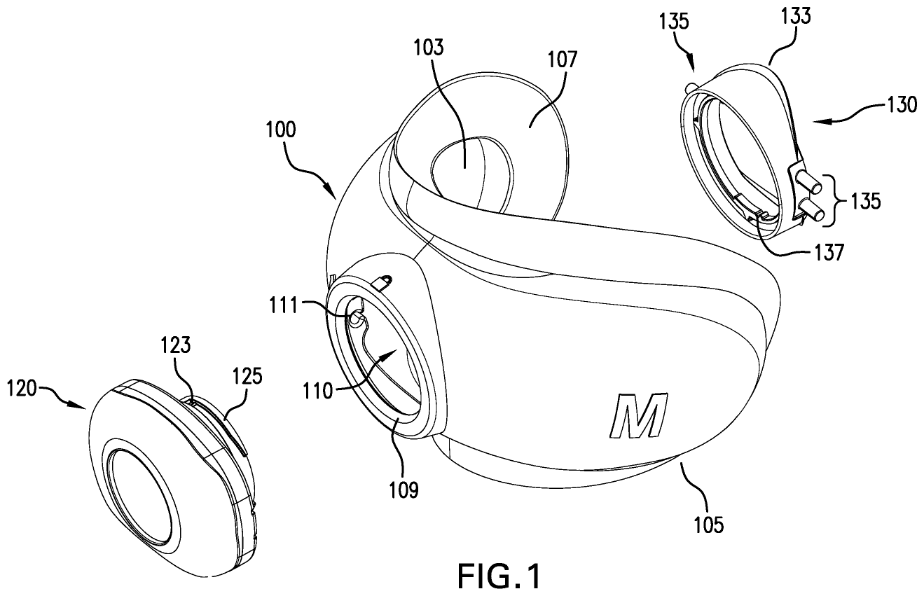

In certain embodiments the chamber body may contain features that further aid in preventing regional collapse, bottoming and transfer of force from one region to another of therapy device. These may be aided by structural elements of the sealing system for connecting the air pump to the chamber, such as by the chamber body support feature 133 shown in FIG. 1. Absent local points of flexibility, a rigid chamber body may experience situations where external pressure could cause a point of high contact pressure for example upon application of a force, by rolling on to a pillow etc., on the device causing a bottoming event or further a rigid chamber body may experience situations where external pressure on the device on one side causes a transfer of force to the opposite side of the device. Events such as these may cause discomfort, dislodging of the device or both.

The chamber body is operably connected to an air pump to produce therapeutic level of negative pressure within the chamber body. The air pump can be of any type to suitable to produce therapeutic level of negative pressure for example positive displacement pumps, impulse pumps, velocity pumps, etc which can include manual squeeze bulbs, rotary pumps, lobe pumps, oscillatory pumps etc. In certain embodiments the air pump comprises a piezoelectric material configured to provide an oscillatory pumping action wherein the oscillatory pumping motion operates at a frequency greater that 500 Hz.

The air pump may be a separate component connected to the chamber body via a hose or tube, or may be configured integrally to the chamber body. The air pump can be connected to the chamber body in any suitable fashion for example an air pump may external the chamber body and connected via a hose or tube, stationary, for example bed-side, or battery powered and wearable by the patient. In certain wearable aspects, the air pump is configured to be integral to the chamber body. For example, the air pump may be configured to insert into a sealable aperture on the chamber body, the air pump tightly fitting through the sealable aperture creating a seal. As used herein a sealable aperture is an opening through an element of the apparatus that can be closed or sealed from one side or the other with another element of the apparatus creating an air-tight or water tight seal.

The seal between the air pump and the sealable aperture of the chamber body comprises a lip seal. As used herein a lip seal is a sealing feature comprising a resilient sealing lip that extends at some angle into the sealable aperture such that when the air pump element is inserted into the sealable aperture, the lip seal makes contact with the air pump housing. This compression of the lip seal during insertion creates an airtight seal between the air pump and the chamber body. The lip seal feature may be an integral, unitary or discrete part of the air pump, the chamber body or both. In certain embodiments the lip seal is provided as a component of the air pump. In a preferred embodiment, however, the lip seal is a unitary feature on the inner circumference of the sealable aperture of the chamber body.

The characteristics of the seal created by this compression of the lip seal can be influenced and optimized by several factors. These characteristics are influenced by material selection and durometer, the length, thickness, taper, contact radius of the lip seal element, as well as the amount of interference between the pump and the lip seal, as described hereinafter. For example, tighter or looser fits can be achieved by decreasing or increasing the clearance between the air pump housing and the sealable aperture, and/or increasing or decreasing the durometer of the lip seal element respectively for example. Other factors that may aid in optimizing the seal between features of the device may also include increasing or decreasing the length of the resilient sealing lip and/or the portion of the resilient sealing lip that actually contacts the air pump. Increasing or decreasing the thickness of the lip seal element may also aid in stiffening or softening the bending moment of the lip seal element respectively.

In preferred embodiments of the invention, the sealing features of the device are optimized to allow for ease of multiple assembly/disassembly events which may include insertion and/or removal of an air pump into a sealable aperture and further, in some embodiments, rotation of the air pump in the sealable aperture as it locks into its assembled and functional orientation. The dimensions of device features and durometer of the flexible sealing elements must be such to accommodate for assembly/disassembly events throughout the lifetime of the device thereby providing sufficient sealing of device components. These dimensions allow for a therapeutic level of pressure to be delivered during therapy with no leakage between device components while not being so tight or overbearing to impede a user's ability to assemble or disassemble device components.

The lip seal design illustrated in the figures is optimized to provide a reliable seal that resists leaking caused by chamber body movement and flexing, but also allows easy assembly/disassembly. Friction between the lip seal and the air pump element is optimized and minimized to allow easy rotation of the pump during assembly/disassembly while also preventing the retainer collar from being dislodged into the chamber during assembly. A locking arrangement such as a bayonet mount to an interior retainer collar is preferably used to secure the pump, ensuring proper positioning between the lip seal element and pump contact surface as well as preventing over-tightening of the connection.

Because the chamber body is typically made of a flexible material, a reinforcing structure may be provided as a retaining-type element to both secure the air pump to the flexible chamber body while also providing structural support to support the chamber itself and/or to isolate the aperture from movement experienced in the flexible chamber during use. As used herein a retaining-type element is defined as a rigid element designed to engage with and secure the air pump element within the aperture of the chamber body. The engagement may be achieved by friction, a snap fitting, a rotatable fitting such as a bayonet-type fitting, etc.

In certain embodiments, the flexible chamber body of a pressure containment structure can be mechanically supported by structure(s) that may be formed as discrete element(s) containing one or more structural members shaped to approximately fit against the interior of the pressure containment structure thereby preventing collapse of the flexible chamber body. The structural elements may also be integrated into a removably securable retaining element.

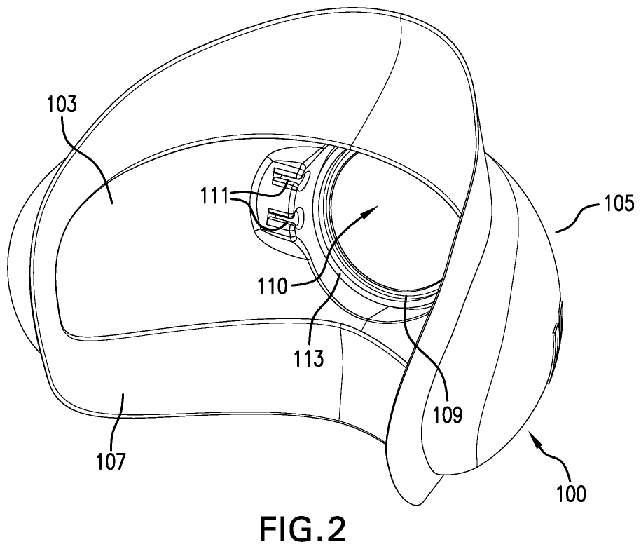

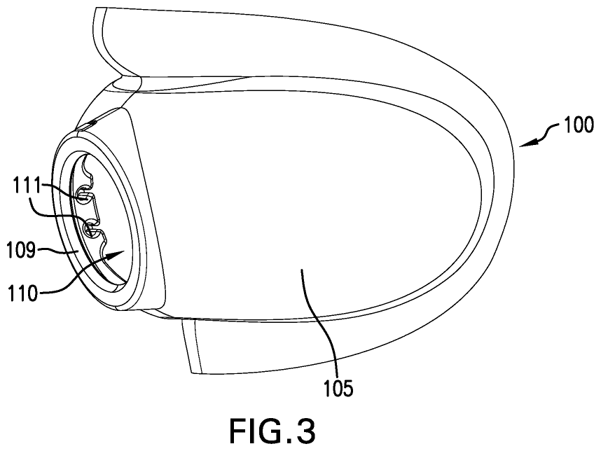

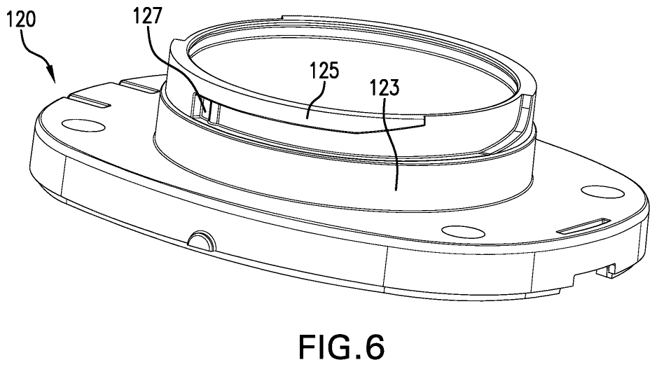

In a preferred embodiment of the device, as seen in FIGS. 1 to 6, the device comprises a chamber body 100 defining an interior volume and having an interior aspect 103 facing the skin surface, and an exterior aspect 105 facing the ambient air. The chamber body comprises a skin interface surface designed to make contact with the perimeter of therapy site of the user 107, a lip seal feature 109, an aperture 110 configured to receive an air pump, recesses 111 (described hereinafter) and retaining element channel 113.

The device further comprises a pump element 120 comprising a first collar interface surface 123 depicted in the figures as comprising a first bayonet connector 125; and a retainer collar 130, comprising chamber body support feature(s) 133, mount points 135, a continuous peripheral edge region 137 that forms a lip seal reinforcing element in use, and a second bayonet connector 139.

The device is assembled by inserting pump element 120 into the aperture 110 of the chamber body 100, and securing the pump element from the inner aspect of the chamber body to the chamber body via engagement with the retainer collar 130. The retainer collar comprises mount points 135 that seat into the retaining element securing recesses 111 of the chamber body. It is preferred that the fit between the retaining element securing recesses 111 and the mount points 135 be tight enough to overcome the insertion forces of the air pump element 120 into the chamber body 100 so that the retainer collar 130 remains integrated in the flexible chamber body 100 when assembled. However, the fit between the retaining element securing recesses 111 and the mount points 135 still permit rapid and easy removal of the retainer collar 130 upon inversion of the flexible chamber body 100 when the air pump element 120 is removed.

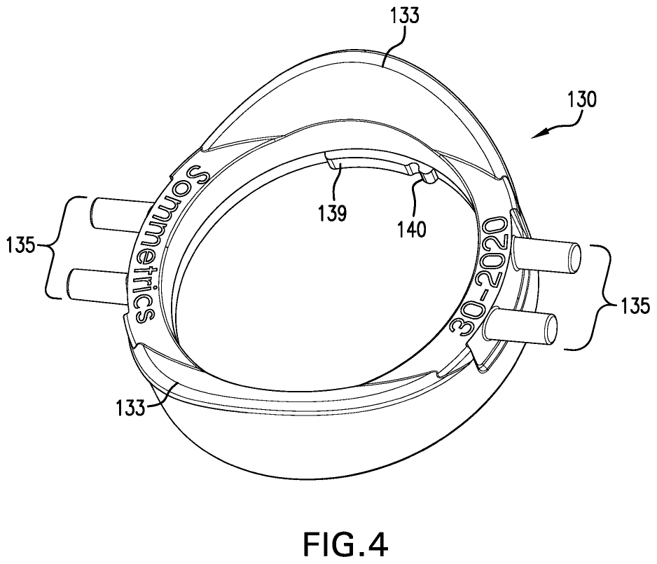

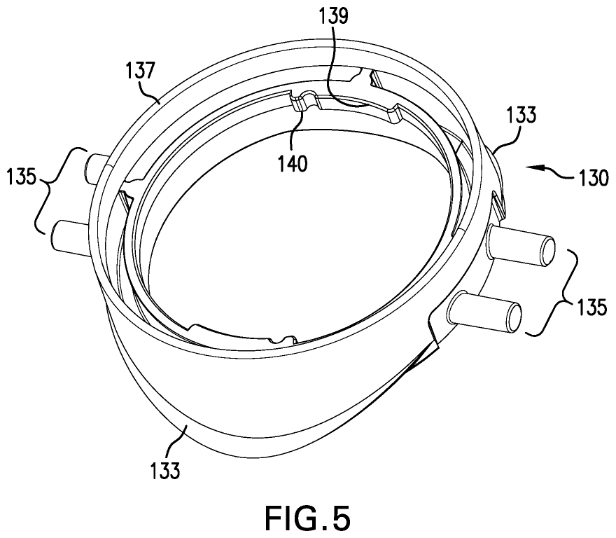

A locking and orientation mechanism may be provided, for example, such that, when the air pump 120 is lined up with the bayonet feature 139 of the retainer collar (FIGS. 4 and 5) and is inserted into and through the sealable aperture 110 and the retainer collar 130 and rotated until the air pump bayonet feature 125 reaches the bayonet stop feature (FIGS. 4 and 5) 140 of the retainer collar. The air pump sealing surface 123 mates with the lip seal 109 of the chamber body 100 creating an air-tight seal.

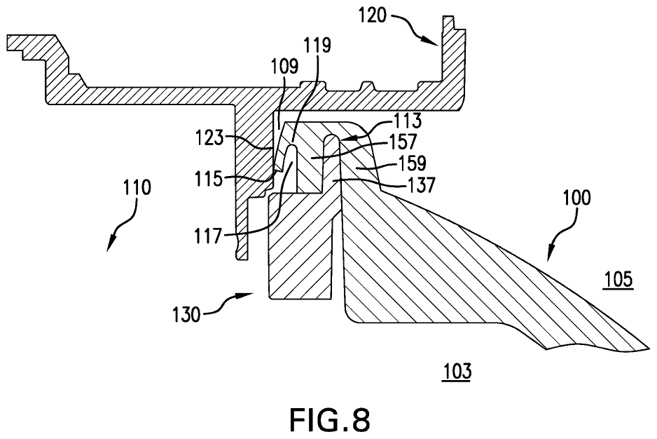

In certain embodiments, the lip seal feature 109 may further substantially isolate the sealing feature from structural loads caused by the chamber body during use by means of one or more structural members. These structural members may be integrated, discreet or unitary. For example, in FIG. 8, the sealable aperture 110 of the chamber body 100 may comprise a resilient sealing lip of lip seal 109 a first recess 117 into which the lip seal element can be deflected by insertion of air pump element 120, the recess comprising an apex 119. The lip seal element may 109 be thinner at the pump housing contact point 115/123, then gradually increasing in thickness as the lip seal element reaches the apex of the first recess 119. The first recess 117 is backed by a first backing support 157, a second recess 113 configured to receive as a supporting structure element the continuous peripheral edge region 137 of the retainer collar 130, and a second backing support 159. Thus, the first backing support 157 and the second backing support 159 define the second recess 113.

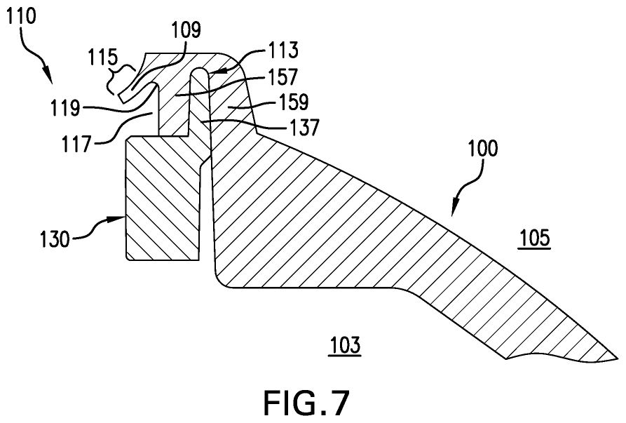

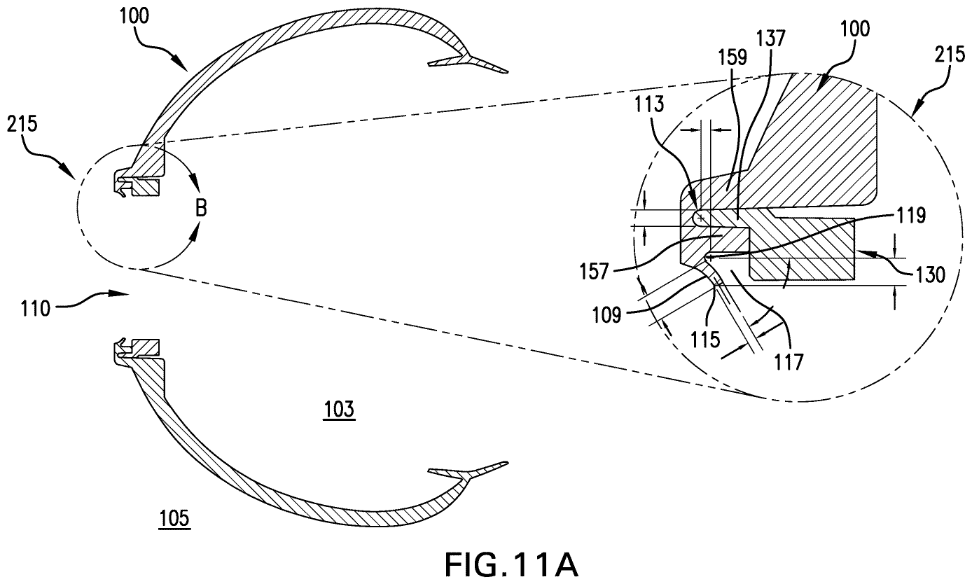

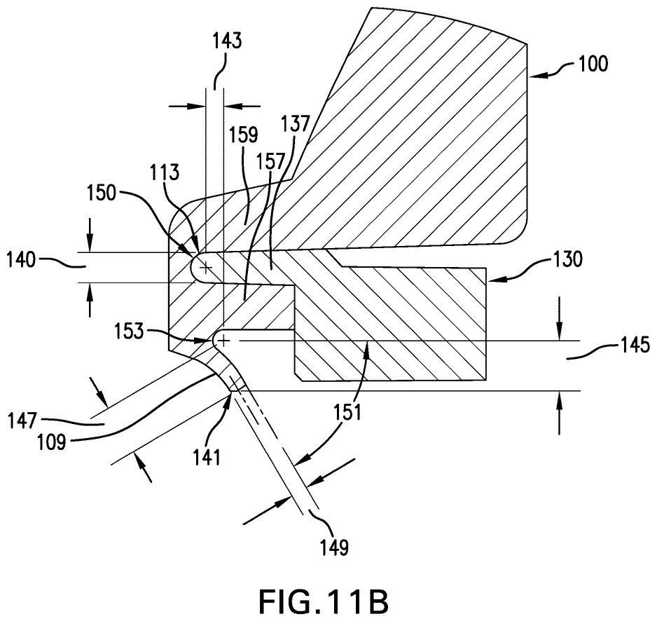

The retainer collar may be in the form of a removably securable rigid or semi-rigid retaining element configured to secure within the flexible chamber body and provide stiffening properties to the aperture, thereby serving to maintain the shape of the aperture and lip seal feature. For example, as seen in FIGS. 5, 7, 8, 11 and 12, the retainer collar 130, comprises an annular continuous peripheral edge region 137 that inserts into second recess 113 of the chamber body 100. For proper operation, the inside diameter of the retainer collar should be seated against the outside diameter of the second channel 113. The protrusion provided by the peripheral edge region 137 will have an insertion depth dimension 143 that extends to at least to the position of the maximum bending moment on the resilient sealing lip of the lip seal 109, and more preferably extends beyond the bending moment of the lip seal as depicted in FIG. 11b.

With engagement and proper seal between the inside diameter of the removably securable retainer collar 130 and the insider diameter of the retaining element channel 11,3 the aperture 110 will be isolated from movement of the flexible chamber body 100 thereby inhibiting seal disruption during use of the device.

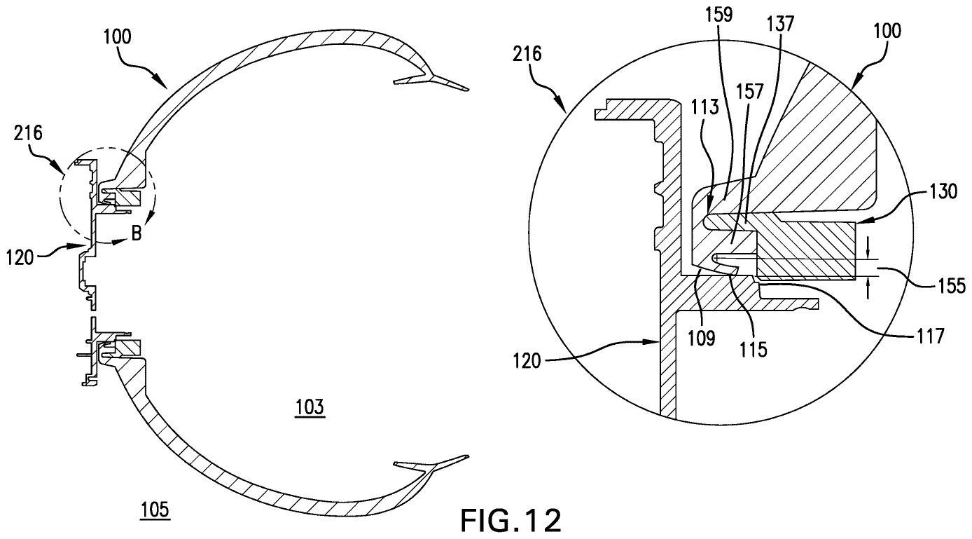

In a preferred embodiment of the invention as seen in FIG. 11b, the flexible chamber body 100 may be formed from a molded silicone rubber of a Shore A durometer of approximately 20-60 and more preferably a Shore A durometer of approximately 30-40 and may have a retaining element channel with width dimension 140 that is approximately 1.03 millimeters in width with retaining element channel with a radius 150 of approximately 0.52 millimeters at its apex, a lip seal feature 109 with a length dimension 147 that is approximately 1.77 millimeters, a width dimension 149 that is approximately 0.60 millimeters thick, with a first recess apex with a radius dimension 153 of approximately 0.4 millimeters that has an angle dimension 151 that is approximate 60 degrees from the outer surface of the retaining element channel into the sealable aperture when the pump is not installed and a pump housing contact sealing surface dimension 141 with a radius of approximately 0.12 millimeters, where when the pump housing is inserted into the sealable aperture FIG. 12, the lip seal feature 109 makes contact with the sealing surface of air pump 115 deflecting inward toward the retainer collar 130 to a compressed dimension 155 of approximately 0.37 millimeters thereby creating a seal such that when a therapeutic level of negative pressure is applied within the chamber, no flow of air passes between the air pump 120 and the lip seal element 109.

In certain aspects of the invention, one or more tangs, tabs and or recesses are present on the chamber body, skin interface and or air pump element of therapy device, which provide one or more guidance feature(s) to ensure a proper orientation of, or mating between one or more device elements. The tangs, tabs and or recesses can be utilized as part of a sensor system to determine various parameters related to use of therapy device. These parameters can include, but are not limited to, compatibility of the particular air pump element with therapy device element (e.g., acting as a recognition feature) and correct placement of the air pump element into the aperture of the chamber body. For example, one or more of these tangs, tabs or recesses can be located on the chamber body as a guidance feature for the air pump element such that a recess on the air pump element or chamber body accepts the tang or tab element on the chamber body or air pump element only when the air pump element is inserted through the sealable aperture in the correct orientation. This list is not meant to be limiting.

As used herein, “user compliance” refers to the patient's adherence to the prescribed usage of a therapy device for example the usage of a device throughout a sleep cycle.

As used herein, “device compliance” refers to the ability of the device or elements of the device to accommodate variation, for example, bending, twisting, compressing and or expanding of the device in response to device application and usage including anatomical variations of the patient.

Aspects of the device may be made of a rigid or substantially rigid material. The skilled artisan will understand that a number of polymers may be used including thermoplastics, some thermosets, and elastomers. Thermoplastic materials become flowing liquids when heated and solids when cooled, they are often capable of undergoing multiple heating/cooling cycles without losing mechanical properties. Thermoset materials are made of prepolymers which upon reaction cure irreversibly into a solid polymer network. Elastomers are viscoelastic materials which exhibit both elastic and viscous properties and can be either a thermoplastic or thermoset. Common thermoplastics include PMMA, cyclic olefin copolymer, ethylene vinyl acetate, polyacrylate, polyaryletherketone, polybutadiene, polycarbonate, polyester, polyetherimide, polysulfone, nylon, polyethylene, and polystyrene. Common thermosets include polyesters, polyurethanes, duroplast, epoxy resins, and polyimides. This list is not meant to be limiting. Functional filler materials such as talc and carbon fibers can be included for purposes of improving stiffness, working temperatures, and part shrinkage.

Aspects of the device may be formed using a number of methods known to those of skill in the art, including but not limited to injection molding, machining, etching, 3D printing, etc. In preferred embodiments, the test device base is injection molded, a process for forming thermoplastic and thermoset materials into molded products of intricate shapes, at high production rates and with good dimensional accuracy. The process typically involves the injection, under high pressure, of a metered quantity of heated and plasticized material into a relatively cool mold—in which the plastic material solidifies. Resin pellets are fed through a heated screw and barrel under high pressure. The liquefied material moves through a runner system and into the mold. The cavity of the mold determines the external shape of the product while the core shapes the interior. When the material enters the chilled cavities, it starts to re-plasticize and return to a solid state and the configuration of the finished part. The machine then ejects the finished parts or products.

Those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims:

What is claimed is:

1. A negative pressure therapy device, comprising
a chamber body comprising
(iv) a skin interface at the periphery of the chamber body configured to provide a skin contact surface with the individual, wherein when the skin interface is in contact with the individual, the chamber body defines an external aspect facing the ambient environment, an internal aspect facing the skin tissue, and an enclosed interior volume overlying the skin tissue that is enclosed by the chamber body,
(v) an aperture through the chamber body,
(vi) a sealing interface at the periphery of the aperture, the sealing interface comprising
a lip seal comprising a resilient sealing lip projecting toward the interior of the chamber body,
a first recess posterior to the resilient sealing lip relative to the aperture,
a first backing support posterior to the first recess relative to the aperture,
a second recess posterior to the backing support relative to the aperture, and
a second backing support posterior to the second recess relative to the aperture,
wherein the lip seal and the first and second backing supports are configured as unitary components of the chamber body;
an air pump configured to insert into the aperture from the external aspect of the chamber body and comprising a first collar interface surface, wherein upon insertion into the aperture the air pump is operably connected to the chamber body to remove air from the enclosed volume when energized, the air pump deflects the resilient sealing lip into the first recess, thereby forming an airtight seal with the chamber body, and the first collar interface surface is accessible from the interior of the chamber body;
a rigid or substantially rigid retainer collar comprising a continuous peripheral edge region configured to insert into the second recess and a second collar interface surface configured to cooperatively engage with the first collar interface surface from within the interior aspect of the chamber body, wherein insertion of the peripheral edge region into the second recess and engagement of the first and second collar interface surfaces reversibly retains the air pump and retainer collar to the chamber body.

2. A negative pressure therapy device according to claim 1, wherein the device is configured and arranged for treating sleep apnea, snoring, or airway obstruction.

3. A negative pressure therapy device according to claim 1, wherein the device is configured and arranged for treating a wound.

4. A negative pressure therapy device according to one of claims 1-3, wherein the lip seal is positioned at or very near to the external aspect of the chamber body.

5. A negative pressure therapy device according to claim 4, wherein the resilient sealing lip contacts the air pump when the air pump is seated into the aperture, but does not contact the retainer collar.

6. A negative pressure therapy device according to one of claims 1-5, wherein the first and second collar interface surfaces cooperate to provide a reversible locking mechanism that orients the air pump relative to the to the retainer collar.

7. A negative pressure therapy device according to claim 6, wherein the first and second collar interface surfaces provide a cooperative twist coupling.

8. A negative pressure therapy device according to claim 6, wherein the first and second collar interface surfaces provide a cooperative bayonet mount.

9. A negative pressure therapy device according to one of claims 1-8, wherein the second recess extends between the first backing support and the second backing support to at least the point of maximum bending moment of the resilient sealing lip.

10. A negative pressure therapy device according to one of claims 1-9, wherein the continuous peripheral edge region is configured to insert into the second recess until the second recess is fully occupied by the continuous peripheral edge region.

11. A negative pressure therapy device according to one of claims 1-10, wherein the air pump and/or the retainer collar comprise mount points configured to engage with receiving points on the chamber body and to secure the pump module to the chamber body in a specific orientation.

12. A method of performing negative pressure therapy on an external surface of an individual, comprising:
mating a negative pressure therapy device of one of claims 1-11 on the external surface to enclose an interior volume overlying skin tissue that is enclosed by the chamber body, and
evacuating air from the interior volume by energizing the air pump.
